(19) United States
(12) Patent Application Publication
(10) Pub. No.: US 2024/0247022 A1
(43) Pub. Date: Jul. 25, 2024

(54) A Process for Synthesis of Nicotinamide Riboside Chloride (NRCL)

(71) Applicant: Bohan & Co AS., Oslo (NO)

(72) Inventors: Ramani Malkannagari, Hyderabad (IN); Raghava Rao Gundapuneni, Hyderabad (IN); Frode Bohan, Snaroya (NO)

(21) Appl. No.: 18/558,779

(22) PCT Filed: May 4, 2022
(86) PCT No.: PCT/EP2022/061950

§ 371 Date: Nov. 3, 2023

(30) Foreign Application Priority Data
May 4, 2021 (IN) 202121020313

(51) Int. Cl. C07H 19/048; C07H 1/00

(52) U.S. Cl. CPC C07H 19/048; C07H 1/00

(57) ABSTRACT

The present invention discloses synthesis of nicotinamide riboside chloride (NRCI). More particularly, the present invention describes cost effective and industrially scalable process for the synthesis of NRCI in amorphous form.

### TECHNICAL FIELD

The present invention relates to synthesis of nicotinamide riboside chloride (NRCI). More particularly, the present invention relates to cost effective and industrially scalable process for the synthesis of NRCI in amorphous form.

### Background and Prior Art

Nicotinamide riboside and its derivatives such as nicotinamide riboside chloride and nicotinamide mononucleotide are the metabolites of nicotinamide adenine dinucleotide (NAD+). As a NAD+ precursor, nicotinamide riboside exhibited enhanced oxidative metabolism and protects against high-fat diet induced obesity in mice, leading to significant interest in nicotinamide riboside and its derivatives. Since nicotinamide riboside is a naturally occurring compound, nicotinamide riboside and its derivatives have great potential as natural, nutritional supplements without causing side effects. One limitation in the commercial exploitation of nicotinamide riboside and its derivatives is that known synthetic method for preparing nicotinamide riboside and its derivatives have several disadvantages, rendering them unsuitable for scaling up for commercial or industrial use.

For example, WO 2007/061798 describes a method for the preparation of nicotinamide riboside and its derivatives via triflate salt form of nicotinamide riboside. Since, the triflate salt form of nicotinamide riboside is not a nutritional supplement, because of its associated toxicity. Therefore, these compounds require an additional step to exchange the triflate anion for another anion that would be pharmaceutically acceptable, using methods such as reverse phase liquid chromatography or ion exchange chromatography thereby escalating the cost of the manufacturing process. Moreover, in view of the labile nature of nicotinamide riboside under the chromatographic conditions; the same could result in less purity and yields due to the side products formation.

WO2007/061798 (Examples 1 and 2) describes preparation of nicotinamide riboside by reaction of 1,2,3,5-tetra-o-acetyl-β-D-ribofuranose with ethyl nicotinate in presence of trimethylsilyl trifluoromethanesulfonate (TMSOTf) in CH₂CL₂ at reflux to generate compound 2′, 3′, 5′-Triacetyl ethyl nicotinate riboside (ethyl 1-[3,4-diacetyloxy-5-(acetyloxymethyl)oxolan-2-yl]-pyridine-3-carboxylate), which is de-acetylated and amidated in methanolic ammonia to provide nicotinamide riboside (NR), followed by purification using reverse HPLC. Although, these examples mention that the product obtained is pure; however, neither purity nor yield is reported. The synthesis is shown in below scheme 1.

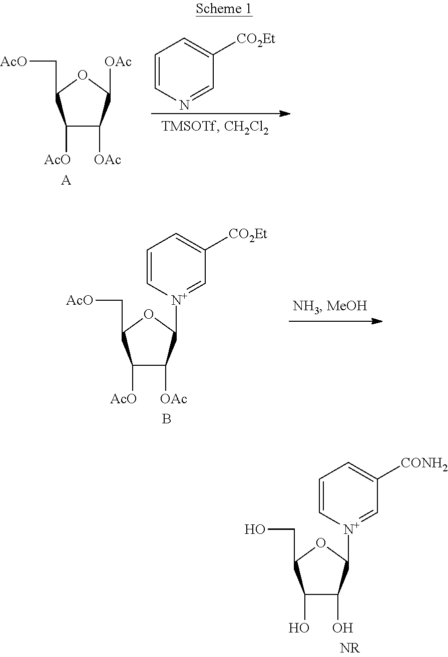

Similar procedure is described in WO2015/014722 (examples 1 to 3, FIG. 3) wherein, the coupling of bis-silylated nicotinamide (prepared from nicotinamide and Trimethylsilyl chloride (TMSCI) in hexamethyldisilazane (HMDS) with ribose tetraacetate in the presence of five equivalents of TMSOTf. In this case also, the conversion to the pharmaceutically acceptable chloride salt requires several additional steps.

TMSOTf-mediated couplings between a nicotinamide derivative and a protected ribose have been reported in Franchetti, P. et al, Bioorg. Med. Chem. Lett. 2004, 14, 4655-4658; Tanimori, S. et al, Bioorg. Med. Chem. Lett. 2002, 12, 1135-1137; and Yang, T. et al, J. Med. Chem. 2007, 50, 6458-6461. These methods have the disadvantage of inevitably resulting in the preparation of the triflate salt by virtue of using TMSOTf as catalyst leading to an additional step of ion exchange. WO2019006262A1 discloses another process for the synthesis of NRCI wherein chlorination of 1,2,3,5-tetra-acetyl-D-ribofuranose to obtain tri-O-acetyl ribofuranosyl chloride is reported to be conducted in presence of HCl dissolved in dioxane using dichloromethane as reaction solvent with optional use of acetyl chloride or HCl dissolved in acetonitrile to obtain chloro derivative; followed by coupling with nicotinamide in presence of tributylamine in acetonitrile to obtain tri-O-acetyl β-nicotinamide riboside chloride, which is further hydrolysed in presence of methanolic ammonia or diethylamine, to obtain nicotinamide riboside chloride. This process involves costly solvents such as 1,4 dioxane and acetonitrile and hence escalates the cost of the total synthesis of nicotinamide riboside chloride thereby use of these solvents makes the process unviable on industrial scale. Deacetylation of triacetyl nicotinamide riboside requires cryogenic conditions (−3° C.) over prolonged reaction times (36 h) using a solution of diethyl amine in methanol. The regeneration of the product from the reaction mass using HCl is sluggish resulting in low to moderate yields.

In another prior art (Journal of Chemical Sciences, 1948, 967), the chlorination of 1,2,3,5-tetra-acetyl-D-ribofuranose to obtain Tri-O-acetyl ribofuranosyl chloride is conducted in presence of ethereal dry HCl at 0° C. However, ether is not a suitable solvent for industrial scale up due to its high volatility and related safety issues.

As is evident from the above, the prior art suffers from lot of disadvantages such as additional steps like ion exchange chromatography for replacement of triflate with suitable counter ion to make nicotinamide riboside suitable for consumption; use of costly solvents in chlorination step makes the process unviable for industrial scale up; requirement of cryogenic conditions for deacetylation process, loss of yield due to incomplete hydrolysis of the acetyl groups coupled with difficulty in purification of nicotinamide riboside chloride using chromatographic techniques.

Therefore, there remains a need in the art to develop a cost effective, highly efficient and industrially scalable processes for the manufacture of nicotinamide riboside chloride.

In the light of the foregoing, the objective of the present invention is to provide an industrially viable and cost-effective process for preparation of nicotinamide riboside chloride.

### SUMMARY OF THE INVENTION

In line with the above objective, the present invention provides a process for preparation of nicotinamide riboside chloride which process comprises;
a) Chlorinating ribose tetraacetate (RTA) by treating with dry HCl either in acetone or in acetonitrile followed by insitu coupling of chloro derivative either with Nicotinamide or with 3-cyanopyridine respectively in presence of an organic base to obtain tri-O-acetyl β-nicotinamide riboside chloride or tri-O-acetyl β-nicotinonitrile riboside chloride respectively; and
b) Deacetylating the tri-O-acetyl β-nicotinamide riboside chloride or the tri-O-acetyl β-nicotinonitrile riboside chloride in presence of HCl in an alcoholic solvent, to afford nicotinamide riboside chloride.

Accordingly, in a preferred aspect, the present invention provides a process for preparation of nicotinamide riboside chloride which process comprises;
a) Chlorinating ribose tetraacetate (RTA) by treating with dry HCl, in acetone followed by insitu coupling of chloro derivative with nicotinamide in presence of an organic base to obtain tri-O-acetyl β-nicotinamide riboside chloride; and
b) Deacetylating the tri-O-acetyl β-nicotinamide riboside chloride in presence of HCl in an alcoholic solvent to afford nicotinamide riboside chloride.

In another preferred aspect, the present invention provides a process for preparation of nicotinamide riboside chloride which process comprises;
a) Chlorinating ribose tetraacetate (RTA) by treating with dry HCl in acetonitrile followed by insitu coupling of chloro derivative with 3-cyanopyridine in presence of an organic base to obtain tri-O-acetyl β-nicotinonitrile riboside chloride; and
b) Deacetylating the tri-O-acetyl β-nicotinonitrile riboside chloride in presence of HCl in an alcoholic solvent to afford nicotinamide riboside chloride.

In yet another preferred aspect, the present invention provides a process for preparation of nicotinamide riboside chloride which process comprises;
a) Preparing tri-O-acetyl β-nicotinonitrile riboside triflate by treating ribose tetraacetate (RTA) with 3-cyanopyridine in the presence of TMSOTf either in dichloromethane or in dichloroethane; and
b) Hydrolyzing followed by deacetylation of tri-O-acetyl β-nicotinonitrile riboside triflate using HCl in ethanol/water to afford nicotinamide riboside chloride.

In another aspect, the nicotinamide riboside chloride obtained by the process of the present invention is in the form of amorphous solid.

### DETAILED DESCRIPTION OF THE INVENTION

The invention will now be described in detail in connection with certain preferred and optional embodiments, so that various aspects thereof may be more fully understood and appreciated. Unless specified otherwise, all technical and scientific terms used herein have the same meaning as commonly understood by one of ordinary skill in the art, to which this invention belongs. To describe the invention, certain terms are defined herein specifically as follows.

Unless stated to the contrary, any of the words, “including”, “includes”, “comprising”, and comprises” mean “including without limitation” and shall not be construed to limit any general statement that it follows to the specific or similar items.

Accordingly, in one embodiment, the present invention provides a process for preparation of nicotinamide riboside chloride which process comprises;
a) Chlorinating ribose tetraacetate (RTA) by treating with dry HCl either in acetone or in acetonitrile followed by insitu coupling of chloro derivative either with nicotinamide or with 3-cyanopyridine respectively in presence of an organic base to obtain tri-O-acetyl β-nicotinamide riboside chloride or tri-O-acetyl β-nicotinonitrile riboside chloride respectively; and
b) Deacetylating the tri-O-acetyl β-nicotinamide riboside chloride or the tri-O-acetyl β-nicotinonitrile riboside chloride in presence of HCl in an alcoholic solvent, to afford nicotinamide riboside chloride.

Accordingly, in a preferred embodiment, the present invention provides a process for preparation of nicotinamide riboside which process comprises;
a) Chlorinating ribose tetraacetate (RTA) by treating with dry HCl in acetone followed by insitu coupling of chloro derivative with nicotinamide in presence of an organic base to obtain tri-O-acetyl β-nicotinamide riboside chloride; and
b) Deacetylating the tri-O-acetyl β-nicotinamide riboside chloride in presence of HCl in an alcohol to afford nicotinamide riboside chloride.

The process is shown in below scheme 2.

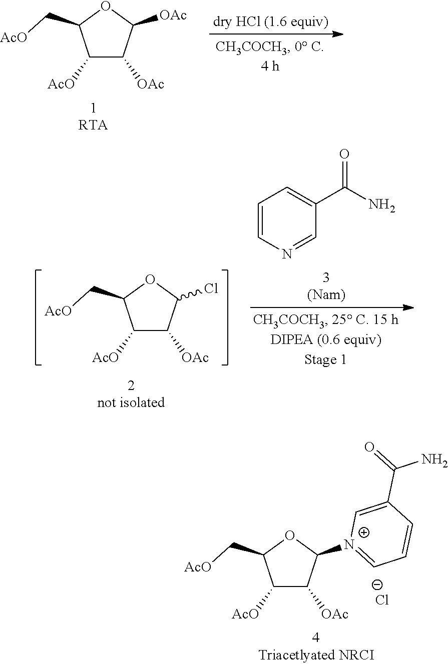

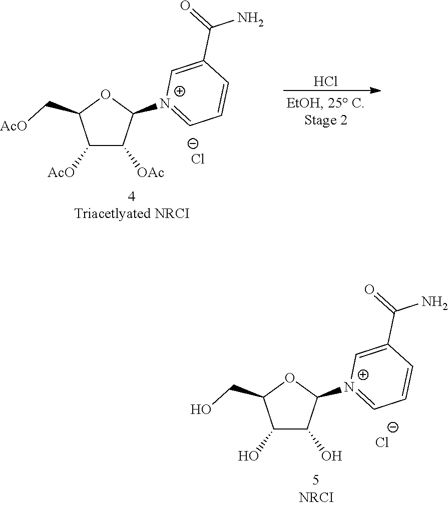 [SMILES: CC(=O)OC[C@H]1O[C@@H]([n+]2cccc(C(N)=O)c2)[C@H](OC(C)=O)[C@@H]1OC(C)=O ; [Cl-] ; Cl ; NC(=O)c1ccc[n+]([C@@H]2O[C@H](CO)[C@@H](O)[C@H]2O)c1 ; [Cl-]]

Chlorination of ribose tetraacetate (RTA) was also studied in other solvents such as t-BuOMe (MTBE), methyl isobutyl ketone (MIBK), tetrahydrofuran (THF), 2-methyltetrahydrofuran (2-methyl-THF), 1,2-dichloroethane, 1,2-dimethoxyethane, diethyl ether, diisopropyl ether, acetonitrile, acetone and 1,4-dioxane. Although, acetone, acetonitrile, Methyl Ethyl Ketone, Methyl Isobutyl Ketone and other ketonic solvents of C4 to C6 were found to be superior to the other solvents studied, however, higher conversion and excellent purity were obtained when acetone is used as solvent. The advantage involved in use of acetone for this transformation is that it is a cost-effective industrial solvent as compared to reported solvents like dioxane for the above transformation. Moreover, if acetone is used for chlorination, the advantage is that the subsequent coupling step with Nicotinamide can be conducted in-situ. Therefore, acetone is the preferred solvent in this transformation. Therefore, in one of the preferred embodiments, the chlorination of 2,3,4,5-tetra-O-acetyl-D-ribose to obtain 1-Chloro-2,3,5-tri-O-acetyl-β-D-ribose and the coupling reaction of 1-Chloro-2,3,5-tri-O-acetyl-β-D-ribose with nicotinamide are carried out in one pot using acetone as a suitable solvent.

In another embodiment, the invention provides a process for preparation of nicotinamide riboside chloride, wherein, the chlorination of 2,3,4,5-tetra-O-acetyl-D-ribose to obtain 1-Chloro-2,3,5-tri-O-acetyl-β-D-ribose and the coupling reaction of 1-Chloro-2,3,5-tri-O-acetyl-β-D-ribose with nicotinamide can be carried out in different pots using acetone as a suitable solvent. In this process the intermediate, 1-Chloro-2,3,5-tri-O-acetyl-β-D-ribose can be isolated and subsequently reacted with nicotinamide.

In an embodiment, the chlorination reaction can be conveniently carried out at a temperature range of 0-5° C.

In another embodiment, the insitu synthesis of tri-O-acetyl β-nicotinamide riboside chloride can be conveniently carried out at a temperature range of 0° C. to 50° C. In another embodiment, the dry HCl or anhydrous HCl used in a solvent can be selected from the group consisting of freshly prepared dry HCl or anhydrous HCl absorbed in a solvent or commercially available dry or anhydrous HCl absorbed in a solvent, wherein, the solvents are selected from acetone, acetonitrile, methanol, ethanol and isopropanol.

In a further embodiment, the molar ratio of Ribose tetraacetate to Nicotinamide is in the ratio of 1:0.5 to 2.0.

For the coupling reaction of 1-Chloro-2,3,5-tri-O-acetyl-β-D-ribose with nicotinamide, various organic bases are studied like triethylamine, triisopropylamine, tributylamine, triisobutylamine, and triisopentylamine, N-methylmorpholine, N-ethylpyrrolidine and tetramethylethylenediamine (TMEDA). However, the bases such as diisopropylethylamine (DIPEA), TBA or N-alkyl pyrrolidine gave the best results.

Accordingly, in another embodiment, the organic base that can be used for the coupling reaction is selected from DIPEA (diisopropylethylamine), TBA or N-alkyl pyrrolidine.

In an embodiment, the alcohol that can be used in the deacetylation of the tri-O-acetyl β-nicotinamide riboside chloride or tri-O-acetyl β-nicotinonitrile riboside chloride or tri-O-acetyl β-nicotinonitrile riboside triflate is selected from the group consisting of methanol, ethanol, isopropyl alcohol, n-propyl alcohol and other C4 to C6 alcoholic solvents.

According to the present invention, the cooled (0° C.) reaction mixture of D-Ribose tetraacetate in acetone was reacted with anhydrous HCl gas under stirring. The resulting mixture was stirred at 0 to 5° C. for another 4-5 h. Upon complete conversion, the nitrogen gas was purged into the reaction mixture for 10-15 min and charged nicotinamide in acetone at 0-5° C. and then added DIPEA (diisopropylethylamine) slowly at the same temperature and the resulting reaction mixture was stirred at a temperature range of 25-50° C. for at least 20 hrs at the same temperature. Upon completion, the reaction mass was cooled to 15 to 20° C. and then filtered to collect the wet cake which was further washed with chilled acetone under suction. The resulting wet cake was dried under vacuum at 30° C. to provide the triacetyl-NRCI.

The advantage involved in use of acetone for the above transformation is that it is a cost-effective industrial solvent compared to reported solvents such as dioxane. Acetone also facilitates clean reaction at lower temperatures and eliminates further work up thereby reduces the costs associated thereof. Acetone plays a dual role as the reaction media as well as purification solvent. Unlike reported processes, this method does not require additional solvents for purification.

In the next stage, deacetylation of triacetyl-NRCI was carried out by reacting with ethanolic or methanolic solution of HCl gas at 0-5° C. under stirring for up to 24 hrs. After complete conversion as indicated by TLC, the reaction mass was filtered off and dissolved in methanol and stirred for 2-3 hrs at 0-5° C. After the completion of the reaction, the product was isolated by filtration and washed with acetone. The resulting wet mass was dried under vacuum at 30° C. for about 2-3 hrs to provide nicotinamide riboside chloride in quantitative yields.

Among various deacetylation agents like HCl, HBr and HI studied for deacetylation reaction, HCl in methanol and ethanol afforded the best results. Of different solvents like methanol, ethanol, isopropanol, t-butanol, diethyl ether, and dioxane investigated; ethanol and methanol afforded the best results. Further, the inventors have also evaluated the efficacy of various alkaline deacetylation agents like methanolic ammonia, ethyl amine, isopropyl amine, diethyl amine, diisopropyl amine, and diisobutyl amine. However, out of all the deacetylation agents studied for this reaction, ethanolic HCl and methanolic HCl gave the best conversion rates out of all the deacetylation agents studied for this conversion.

The deacetylation reaction according to the present invention does not require any cryogenic conditions unlike the prior arts and can be easily conducted at 0-5° C. to afford NRCI in quantitative yields. Deacetylation reaction using cryogenic conditions (−5 to −7° C.) [Lee J. Churchil H. Choi W-B, Lynch J E. Roberts F E. Volante R P, Reider P J. Chem Commun. 1999:729-730. doi: 10.1039/a809930 h] was a limitation in the prior art methods as the application of cryogenic conditions for industrial scale is difficult. Therefore, the present invention simplifies the deacetylation process by employing anhydrous HCl in alcohols such as methanol or ethanol as a solvent and the reaction was performed at 0-5° C. Also, previous methods involve the use of anhydrous ammonia or dialkyl amines as deacetylating agents, in which acetamide or dialkyl amide used to be the by-products. Therefore, tedious work up and purification methods were required to remove acetamide or dialkyl amine and other by-products. However, in the present method, anhydrous HCl was used as a deacetylating agent and hence the by-product formed during the deacetylation is ethyl acetate, which is volatile and environmentally friendly solvent and thus easier to remove from the final product by simple distillation or evaporation.

Further, purification by chromatographic techniques was another challenge in the synthesis of NRCI, which is solved by purification using readily available solvent, such as methanol or ethanolat 0-5° C.

In another preferred embodiment, the present invention provides a process for the preparation of nicotinamide riboside chloride which process comprises;
a) Chlorinating ribose tetraacetate (RTA) by treating with dry HCl at 0° C. in acetonitrile followed by insitu coupling of chloro derivative with 3-cyanopyridine in the presence of an organic base to obtain tri-O-acetyl β-nicotinonitrile riboside chloride;
b) Deacetylating the tri-O-acetyl β-nicotinonitrile riboside chloride in presence of HCl/an alcohol, to afford the β-nicotinamide riboside chloride.

The process according to this embodiment is shown in scheme 3.

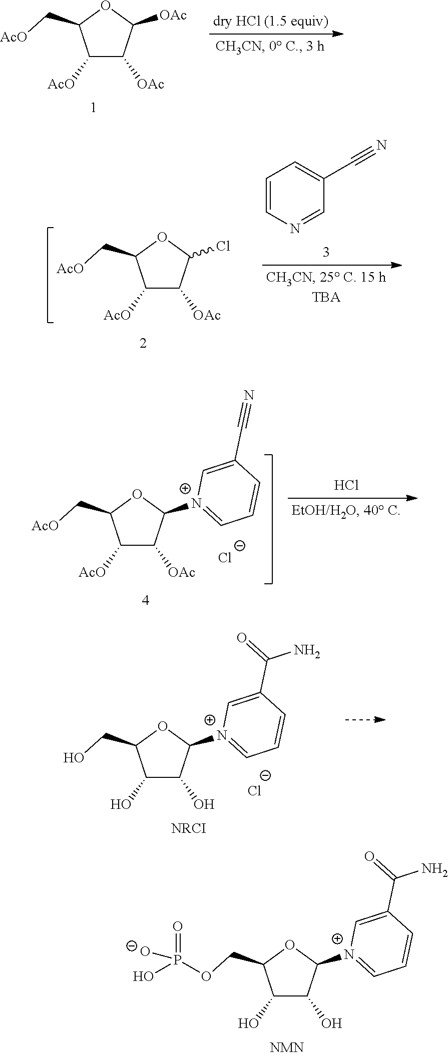

According to the above scheme 3, chlorination of 2,3,4,5-tetra-O-acetyl-D-ribose was conducted by treating with dry HCl at 0-5° C., in presence of acetonitrile. The advantage involved in use of acetonitrile is that the subsequent coupling step with 3-cyanopyridine can be conducted in-situ. Therefore, acetonitrile is the preferred solvent in this transformation according to scheme 3.

In the second step, the chloro derivative was coupled with 3-cyanopyridine in presence of acetonitrile in an organic base selected from TBA or DIPEA to obtain tri-O-acetyl β-nicotinonitrile riboside chloride.

In the third step, the tri-O-acetyl β-nicotinonitrile riboside chloride undergoes deacetylation as well as hydrolysis in presence of HCl in an alcoholic solvent such as methanol, ethanol or isopropanol (Hydrochloric acid in methanol, ethanol or isopropanol), at 40° C. to afford NRCI in high yields.

In yet another preferred embodiment, the invention provides a process for the preparation of nicotinamide riboside chloride which process comprises;
a) Preparing tri-O-acetyl β-nicotinonitrile riboside triflate by treating ribose tetraacetate (RTA) with 3-cyanopyridine in the presence of TMSOTf either in dichloromethane or dichloroethane; and
b) Hydrolyzing followed by deacetylation of tri-O-acetyl β-nicotinonitrile riboside triflate using HCl in ethanol/water to afford nicotinamide riboside chloride.

The process according to this embodiment is shown in scheme 4.

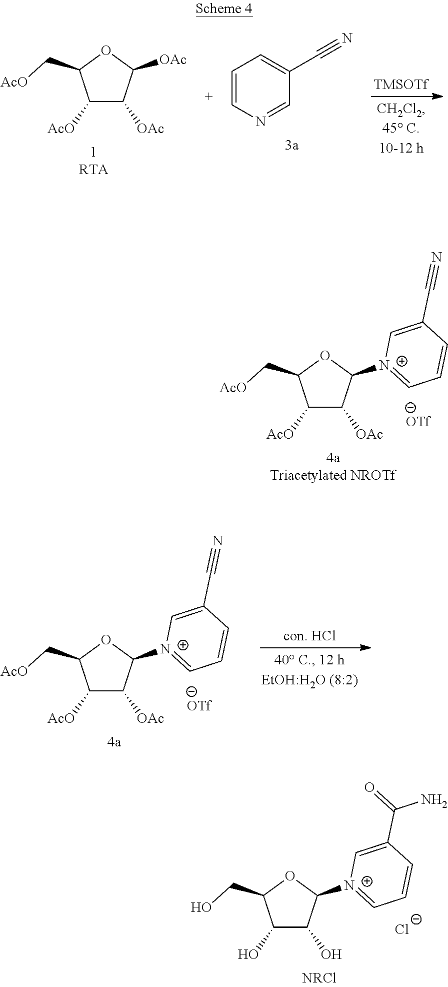

According to the above method, 1,2,3,5-tetra-O-acetyl-β-D-ribofuranose was reacted with 3-cyanopyridine in dry dichloromethane under stirring at room temperature and then a solution of TMSOTf was added slowly at room temperature. After complete addition, the mixture was heated at 45° C. under reflux for 10-12 h.

The progress of the reaction was monitored by TLC and after completion of the reaction, the solvent was removed and the crude was extracted with 1:1 MeOH/hexane and tri-O-acetyl β-nicotinonitrile riboside triflate was recovered from the methanol layer as a colorless liquid.

The solution of tri-O-acetyl β-nicotinonitrile riboside triflate was hydrolysed and deacetylated simultaneously by treating with HCl (40 mmol) in EtOH:H2O (1:1) at 40° C. for 12 h. After completion, the solvent was removed under reduced pressure and the residue was diluted with acetone and the solid was filtered and dried to give crude NRCI, which was purified from acetone to give the pure amorphous NRCI. In an embodiment, the nicotinamide riboside chloride thus obtained by the processes of the present invention, as shown in scheme 2, scheme 3 and scheme 4, is in the form of amorphous solid.

Thus, the processes as described in the present invention offers several advantages like improved yields, cleaner reaction profiles and enables readily available low-cost industrial solvents like acetone, acetonitrile, methanol, ethanol and isopropanol in the synthetic process as well as in the purification stage, thereby making the total synthesis cost-effective and industrially scalable.

The following examples are presented to further explain the invention with experimental conditions, which are purely illustrative and are not intended to limit the scope of the invention.

### EXAMPLES

### Example 1

Stage 1 Part-A: Chlorination of D-Ribose tetraacetate (RTA) using anhydrous HCl

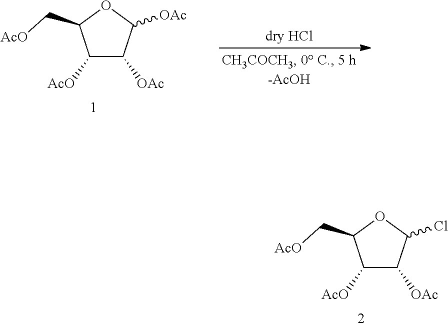 [SMILES: CC(=O)OC[C@H]1OC(OC(C)=O)[C@H](OC(C)=O)[C@@H]1OC(C)=O ; "dry HCl" ; CC(=O)OC[C@H]1OC(Cl)[C@H](OC(C)=O)[C@@H]1OC(C)=O]

To a stirred solution of D-Ribose tetraacetate (100 g) in 250 mL acetone (2.5 vol) in 1 L round bottom flask at room temperature. The solution was cooled to 0° C. and then passed anhydrous HCl gas (18.3 g, 1.6 equiv) into the reaction mixture over 30 min. The resulting mixture was stirred at 0-5° C. for another 4-5 hrs. The progress of the reaction was monitored by TLC (1:1, EtOAc:hexane) and also by GC (IPC Limit: content of RTA by GC is NMT 5%).

### Stage 1 Part-B: Synthesis of tri-O-acetyl β-nicotinamide riboside chloride

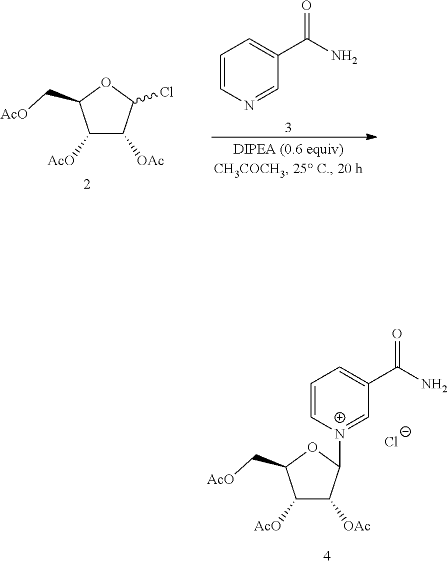

Upon complete conversion of stage 1 Part-A, the nitrogen gas was purged into the reaction mixture for 10-15 min and charged nicotinamide (38.4 g, 1.0 equiv) in 100 mL acetone (1 vol) at 0-5° C. and then added DIPEA (40.6 g, 1.0 equiv) slowly at the same temperature. The resulting mixture was stirred at 25-30° C., over 20 hrs at the same temperature. The progress of the reaction was monitored by TLC (1:9, MeOH:DCM) and also by HPLC (IPC Limit: content of nicotinamide by HPLC is NMT 2%). Upon completion, the reaction mass was cooled to 0° C. and then filtered off to collect the wet cake which was further washed with chilled acetone (500 mL) under suction. The resulting wet cake was dried under vacuum at 30° C. for 2-3 hrs to provide the desired triacetyl-NRCI.

Yield: wet weight: 57 g; dry weight: 50 g; theoretical Yield: 130 g

Melting range: 136-142° C.

¹H NMR (400 MHZ, D₂O) δ 9.45 (s, 1H), 9.22 (d, J=6.3 Hz, 1H), 9.01 (d, J=8.1 Hz, 1H), 8.30 (dd, J=7.9, 6.4 Hz, 1H), 6.61 (d, J=3.8 Hz, 1H), 5.58 (dd, J=5.3, 4.0 Hz, 1H), 5.46 (t, J=5.4 Hz, 1H), 4.95-4.88 (m, 1H), 4.54 (d, J=1.9 Hz, 2H), 2.20-2.05 (m, 9H).

¹³C NMR (101 MHZ, D₂O) δ 173.29, 172.40, 172.34, 165.47, 146.19, 143.08, 140.40, 135.24, 134.14, 128.56, 97.28, 82.56, 76.33, 69.32, 62.57, 20.15, 19.81, 19.78.

HRMS calc for C₁₇H₂₁N₂O₈: 381.1302; found 381.1310

### Stage 2: Deacetylation of triacetyl-NRCI using anhydrous HCl in EtOH

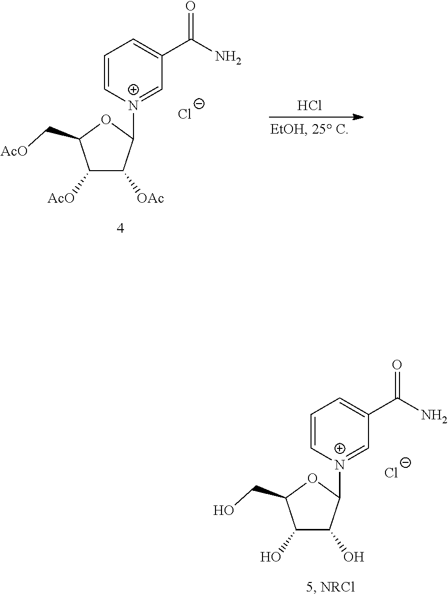 [SMILES: CC(=O)OC[C@H]1OC([n+]2cccc(C(N)=O)c2)[C@H](OC(C)=O)[C@@H]1OC(C)=O ; [Cl-] ; Cl ; NC(=O)c1ccc[n+](C2O[C@H](CO)[C@@H](O)[C@H]2O)c1 ; [Cl-]]

To a stirred solution of triacetyl-NRCI (100 g) in 300 mL ethanol (3 vol) in 1 L round bottom flask at 0° C. was added anhydrous HCl gas (28.3 g, 3 equiv). The resulting solution was stirred at 25° C., over 24 h. After complete conversion as indicated by TLC (2:8, MeOH:DCM), the reaction mass was filtered off and then dissolved in acetone (300 mL); stirred for 2-3 hrs at 25° C. and then filtered and washed with acetone (100 mL) The resulting wet mass was dried under vacuum at 30° C., over a period of 2-3 hrs to yield NRCI as an amorphous solid.

Yield: wet weight 53 g; dry weight: 47 g; theoretical yield: 69 g

Melting range: 125-128° C.

¹H NMR (400 MHZ, D₂O) δ 9.53 (s, 1H), 9.20 (d, J=6.3 Hz, 1H), 9.04-8.77 (m, 1H), 8.21 (dd, J=8.0, 6.4 Hz, 1H), 6.18 (d, J=4.4 Hz, 1H), 4.43 (dt, J=7.2, 4.0 Hz, 2H), 4.29 (t, J=4.7 Hz, 1H), 3.98 (dd, J=12.9, 2.9 Hz, 1H), 3.83 (dd, J=12.9, 3.6 Hz, 1H).

¹³C NMR (101 MHZ, D₂O) δ 165.76, 145.58, 142.59, 140.36, 133.91, 128.30, 99.86, 87.59, 77.37, 69.69, 60.08.

HRMS calc for C₁₁H₁₅N₂O₅:255.1023; found 255.0982

### Example 2

Chlorination of D-Ribose tetraacetate (RTA) by Purging with HCl Stage I:

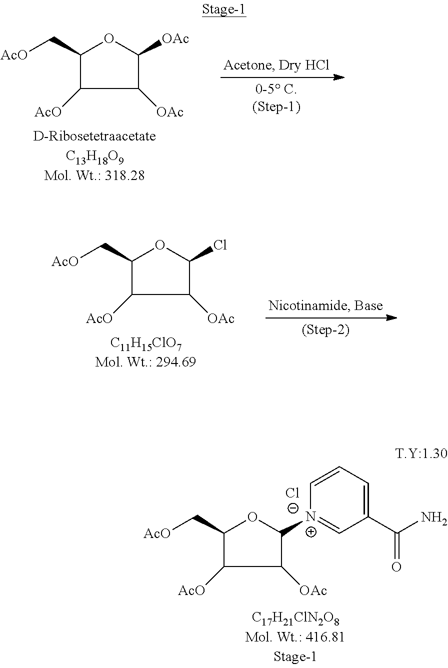

### Synthesis Process for Stage I:

To a clean and dry RBF at 25-30° C., charged acetone (volume: 250 ml) lot-1 at 25-30° C. at R.T, under stirring and mild nitrogen atmosphere. Cooled the acetone to 0-5° C. under mild nitrogen. Weighed the reaction mass (wt.: 196 g), purged the dry hydrochloric acid, into the reaction mass till it gains 18.3 g of HCl weight, below 5° C. Weighed the reaction mass (wt: 214.34 g) (HCl assay is 8.4%). Charged D-ribose tetraacetate (D-RTA) (wt.: 100 g) at 0-5° C. Maintained the reaction mass for additional 15 min at 0-5° C. Warmed the reaction to 8-10° C. and maintained the reaction mass for 4 hrs. Note: Reaction mass becomes clear solution after 30 mins. The sample was analysed by GC for starting material content. (Note: D-RTA content below 5%).Cooled the reaction mass to 0-5° C. and purged moderate nitrogen for 15 min. Charged Nicotinamide(38.27 gm or 1.0 equivalent of D-RTA) at 0-5° C. Added DIPEA, (wt.: 27.61 g or 0.68 equivalent) lot-1 at 0-5° C. Warmed the reaction to 8-10° C. and maintained the reaction mass for 15 min. Warmed the reaction to 38-43° C. and maintained the reaction mass for 16-18 hrs. The sample was analysed by HPLC for product content, Nicotinamide riboside acetate. 16 hrssample analysis showed product purity: 33-35%; Nicotinamide: 36-38%; unknown impurity: 21-23%. Cooled the reaction mass to 15-20° C. Chargedbase lot-2 (wt.: 15 g or 0.37 equivalent) at 15-20° C. (Note: Base quantity is calculated based on unreacted nicotinamide). The reaction mass was stirred continuouslyfor additional 1-2 hrs at 15-20° C. Filtered the mass and washed the bed with 0.5 vol of acetone (volume: 50 ml) lot-2. Suck dried the bed with help of vacuum for 15-20 min. Unloaded the material in round bottom flask and dried the material under vacuum for 2 hrs at 30-35° C. and checked the LOD content as well as HPLC purity.

Yield of Nicotinamide riboside triacetate chloride (NRT-Cl): 50 g as dry wt. HPLC purity of Nicotinamide riboside acetate: 97.05%

### Stage II:

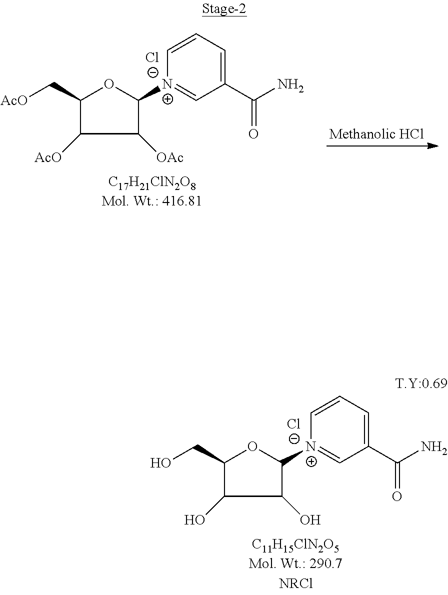

### Synthesis Process for Stage II:

To a clean and dry RBF at 25-30° C., charged Methanol (volume: 250 ml or 5 volume) lot-1 at 25-30° C. Cooled the methanol to 0-5° C. Weighed the reaction mass (wt.: 198 g). Purged the dry hydrochloric acid into the reaction mass till it gains 18.87 g or 4.3 equivalent of HCl weight into the reaction mass below 5° C. Weighed the reaction mass (wt.: 216.86 g) (HCl Assay is 10.4%). Charged Nicotinamide riboside triacetate chloride (NRT-Cl; wt.: 50 g) at 0-5° C. as obtained from Stage I. Maintained the reaction mass for 10-12 hrs at 0-5° C. (Note: Reaction mass becomes clear solution after 2-3 hrs and solid reformed after 6-7 hrs). The sample was analysed by HPLC for starting material content, NRT-Cl. (Note: NRT-Cl content should be below 1%). Filtered the solid and washed the bed with chilled Methanol (25 ml or 0.5 volume) lot-2 under nitrogen. (Note: Nicotinamide riboside chloride (NRCI): 92-95% and Nicotinamide: 2-3%).

### Purification:

Charged chilled Methanol (75 ml or 1.5 volume) lot-3 at 0-5° C. Charged NRCI at 0-5° C. and Stirred for 1-2 hrs at 0-5° C. Filtered the solid and washed the bed with chilled Methanol ((25 ml or 0.5 volume) lot-4 under nitrogen. Suck dried the bed under vacuum for 15-20 min. Unloaded the amorphous material in round bottom flask and dried the material under vacuum for 2 hrs at 30-35° C. (Note: In case if HPLC purity does not meet the specification, then purification step needs to be repeated).

Yield of Nicotinamide riboside chloride: 23.5 g dry wt.

HPLC purity of Nicotinamide riboside chloride: 98.59%

### Example 3

### Chlorination of D-Ribose Tetraacetate (RTA) by Using HCl Cylinder Stage I:

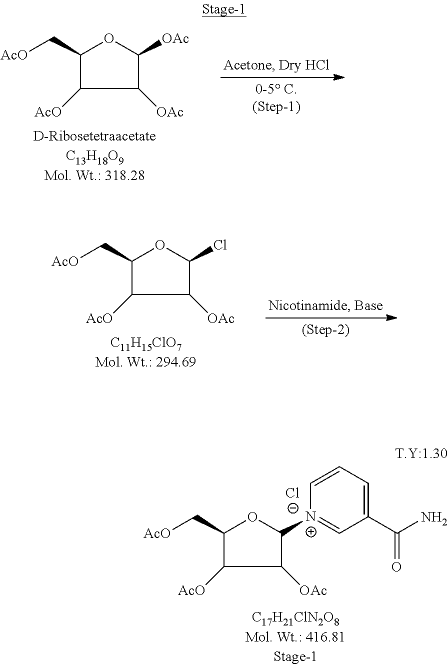

### Synthesis Process for Stage I:

To a clean and dry RBF at 25-30° C., charged acetone (volume: 250 ml) lot-1 at 25-30° C. at R.T, under stirring and mild nitrogen atmosphere. Cooled the acetone to 0-5° C. under mild nitrogen. Weighed the reaction mass (wt.: 196 g). Passed the dry hydrochloric acid from HCl cylinder into the reaction mass till it gains 18.3 g of HCl weight into the reaction mass below 5° C. Weighed the reaction mass (wt: 214.34 g) (HCl assay is 8.4%). Charged D-ribose tetraacetate (D-RTA) (wt.: 100 g) at 0-5° C. Maintained the reaction mass for additional 15 min at 0-5° C. Warmed the reaction to 8-10° C. and maintained the reaction mass for 4 hrs. (Note: Reaction mass becomes clear solution after 30 mins). The sample was analysed by GC for starting material content. (Note: D-RTA content below 5%). Cooled the reaction mass to 0-5° C. and purged moderate nitrogen for 15 min. Charged Nicotinamide (38.27 gm or 1.0 equivalent of D-RTA) at 0-5° C. Added DIPEA, (wt.: 27.61 g or 0.68 equivalent) lot-1 at 0-5° C. Warmed the reaction to 8-10° C. and maintained the reaction mass for 15 min. Warmed the reaction to 38-43° C. and maintained the reaction mass for 16-18 hrs. The sample was analysed by HPLC for product content, Nicotinamide riboside acetate. 16 hr sample analysis showed product purity: 33-35%; Nicotinamide: 36-38%; unknown impurity: 21-23%. Cooled the reaction mass to 15-20° C. Charged base lot-2 (wt.: 15 g or 0.37 equivalent) at 15-20° C. (Note: Base quantity is calculated based on unreacted nicotinamide). Stirring of the mass was continued for additional 1-2 hrs at 15-20° C. Filtered the mass and washed the bed with 0.5 vol of acetone (volume: 50 ml) lot-2. Suck dried the bed with help of vacuum for 15-20 min, unloaded the amorphous material in round bottom flask and dried the material under vacuum for 2 hrs at 30-35° C. and checked the LOD content as well as HPLC purity.

Yield of Nicotinamide riboside triacetate chloride (NRT-Cl): 51 g as dry wt.

HPLC purity of Nicotinamide riboside acetate: 96.05%

### Stage II:

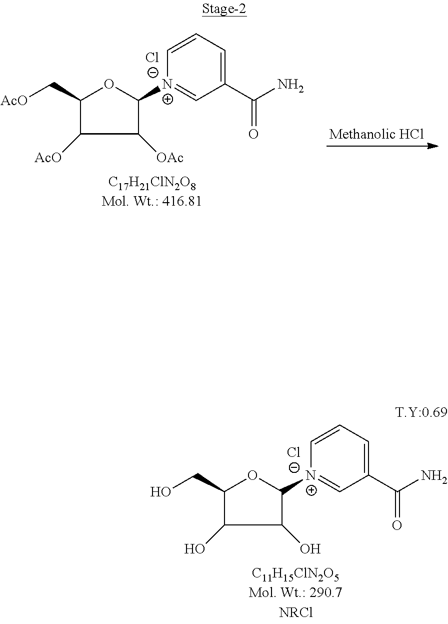

### Synthesis Process for Stage II:

To a clean and dry RBF at 25-30° C., charged Methanol (volume: 250 ml or 5 volume) lot-1 at 25-30° C. Cooled the methanol to 0-5° C. Weighed the reaction mass (wt.: 198 g). Passed the dry hydrochloric acid from HCl cylinder to the reaction mass till it gains 18.87 g or 4.3 equivalent of HCl weight into the reaction mass below 5° C. Weighed the reaction mass (wt.: 216.86 g) (HCl Assay is 10.4%). Charged Nicotinamide riboside triacetate chloride (NRT-Cl; wt.: 51 g) at 0-5° C. as obtained from Stage I. Maintained the reaction mass for 10-12 hrs at 0-5° C. (Note: Reaction mass becomes clear solution after 2-3 hrs and solid reformed after 6-7 hrs). The sample was analysed by HPLC for starting material content, NRT-Cl. (Note: NRT-Cl content should be below 1%). Filtered the solid and washed the bed with chilled Methanol (25 ml or 0.5 volume) lot-2 under nitrogen. (Note: Nicotinamide riboside chloride (NRCI): 92-95% and Nicotinamide: 2-3%).

### Purification:

Charged chilled Methanol (75 ml or 1.5 volume) lot-3 at 0-5° C. Charge NRCI at 0-5° C. and Stirred for 1-2 hrs at 0-5° C. Filtered the solid and washed the bed with chilled Methanol (25 ml or 0.5 volume) lot-4 under nitrogen. Suck dried the bed under vacuum for 15-20 min, unloaded the amorphous material in round bottom flask and dried the material under vacuum for 2 hrs at 30-35° C. (Note: In case if HPLC purity does not meet the specification, then purification step needs to be repeated till to get required HPLC purity).

Yield of Nicotinamide riboside chloride: 24.4 g as dry wt.

HPLC purity of Nicotinamide riboside chloride: 98.43%

### Example 4

### Use of Commercially Available Methanolic-HCl in Stage II

### Stage I:

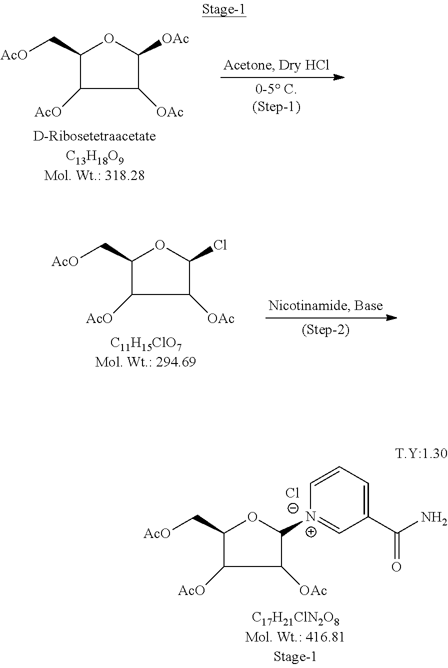

### Synthesis Process for Stage I:

To a clean and dry RBF at 25-30° C., charged acetone (volume: 250 ml) lot-1 at 25-30° C. at R.T, under stirring and mild nitrogen atmosphere. Cooled the acetone to 0-5° C. under mild nitrogen. Weighed the reaction mass (wt.: 196 g). Passed the dry hydrochloric acid from HCl cylinder into the reaction mass till it gains 18.3 g of HCl weight into the reaction mass below 5° C. Weighed the reaction mass (wt: 214.34 g) (HCl assay is 8.4%). Charged D-ribose tetraacetate (D-RTA) (wt.: 100 g) at 0-5° C. Maintained the reaction mass for additional 15 min at 0-5° C. Warmed the reaction to 8-10° C. and maintained the reaction mass for 4 hrs. (Note: Reaction mass becomes clear solution after 30 mins. The sample was analysed by GC for starting material content. Note: D-RTA content below 5%). Cooled the reaction mass to 0-5° C. and purged moderate nitrogen for 15 min. Charged Nicotinamide (38.27 gm or 1.0 equivalent of D-RTA) at 0-5° C. Added DIPEA, (wt.: 27.61 g or 0.68 equivalent) lot-1 at 0-5° C. Warmed the reaction to 8-10° C. and maintained the reaction mass for 15 min. Warmed the reaction to 38-43° C. and maintained the reaction mass for 16-18 hrs. The sample was analysed by HPLC for product content, Nicotinamide riboside acetate. 16 hr sample analysis showed product purity: 33-35%; Nicotinamide: 36-38%; unknown impurity: 21-23%. Cooled the reaction mass to 15-20° C. Charged base lot-2 (wt.: 15 g or 0.37 equivalent) at 15-20° C. (Note: Base quantity is calculated based on unreacted nicotinamide).Continued to stir the mass for additional 1-2 hrs at 15-20° C. Filtered the mass and washed the bed with 0.5 vol of acetone (volume: 50 ml) lot-2. Suck dried the bed with help of vacuum for 15-20 min. Unloaded the material in round bottom flask and dried the material under vacuum for 2 hrs at 30-35° C. and checked the LOD content as well as HPLC purity.

Yield of Nicotinamide riboside triacetate chloride (NRT-Cl): 51 g as dry wt.

HPLC purity of Nicotinamide riboside acetate: 96.3%

### Stage II:

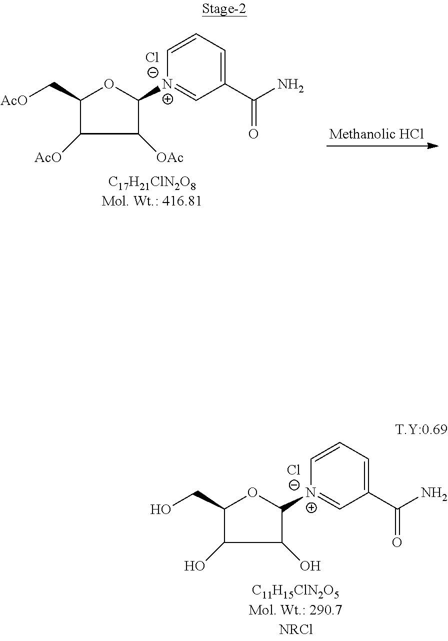

### Synthesis Process for Stage II:

To a clean and dry RBF at 25-30° C., charged readily available Methanol-HCl (volume: 262 ml or 5.2 volume) lot-1 at 25-30° C. Cooled the methanol-HCl to 0-5° C. Weighed the reaction mass (wt.: 216.86 g; HCl Assay is 10.4%). Charged Nicotinamide riboside triacetate chloride (NRT-Cl; wt.: 51 g) at 0-5° C. as obtained from Stage I. Maintained the reaction mass for 10-12 hrs at 0-5° C. (Note: Reaction mass becomes clear solution after 2-3 hrs and solid reformed after 6-7 hrs. The sample was analysed by HPLC for starting material content, NRT-Cl. Note: NRT-Cl content should be below 1%). Filtered the solid and washed the bed with chilled Methanol (25 ml or 0.5 volume) lot-2 under nitrogen. (Note: Nicotinamide riboside chloride (NRCI): 92-95% and Nicotinamide: 2-3%).

### Purification:

Charged chilled Methanol (75 ml or 1.5 volume) lot-3 at 0-5° C. Charge NRCI at 0-5° C. and Stirred for 1-2 hrs at 0-5° C. Filtered the solid and washed the bed with chilled Methanol (25 ml or 0.5 volume) lot-4 under nitrogen. Suck dried the bed under vacuum for 15-20 min. Unloaded the amorphous material in round bottom flask and dried the material under vacuum for 2 hrs at 30-35° C. (Note: In case if HPLC purity does not meet the specification, then the above purification step is required to be repeated till the required purity is achieved).

Yield of Nicotinamide riboside chloride: 24.5 g as dry wt.

HPLC purity of Nicotinamide riboside chloride: 98.30% and above

### Example 5

### Synthesis of NRCI Using 3-Cynopyridine

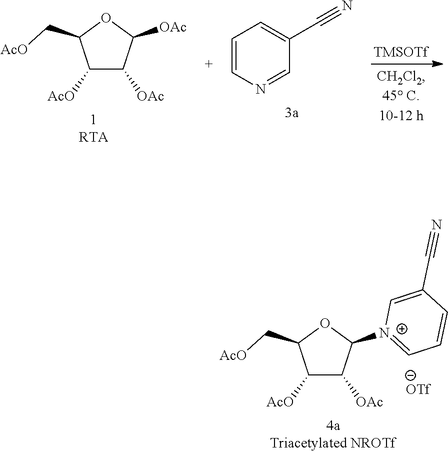 [SMILES: "TMSOTf" ; CC(=O)OC[C@H]1O[C@@H](OC(C)=O)[C@H](OC(C)=O)[C@@H]1OC(C)=O ; N#Cc1cccnc1 ; CC(=O)OC[C@H]1O[C@@H]([n+]2cccc(C#N)c2)[C@H](OC(C)=O)[C@@H]1OC(C)=O]

A mixture of 1,2,3,5-tetra-O-acetyl-β-D-ribofuranose (3.18 g, 10 mmol) and 3-cyanopyridine (1.56, 15 mmol) in dry dichloromethane (100 mL) was stirred under argon atmosphere at room temperature and then a solution of TMSOTf (2.22 g, 10 mmol) was added slowly using a syringe at room temperature. After complete addition, the mixture was heated at 45° C. under reflux for 10-12 h. The progress of the reaction was monitored by TLC (5:0.3:0.05 EtOAc/MeOH/triethylamine eluent) visualized the TLC plate with 10% H₂SO₄/MeOH. After complete disappearance of starting ribose tetraacetate and the appearance of a product as a single UV active spot, the solvent was removed and extracted the crude with 1:1 MeOH/hexane (5×100 mL). The n-hexane layer was separated and the methanol layer was concentrated to give the product 4a in 3.23 g, 89% yield as a colorless liquid.

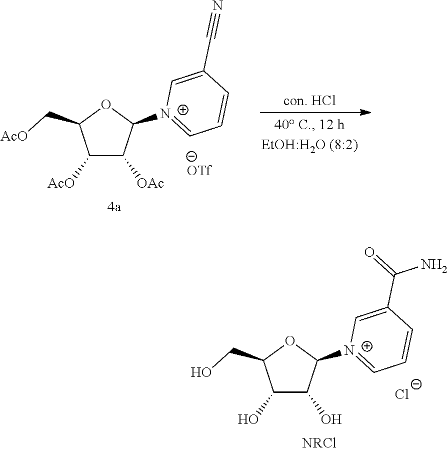 [SMILES: CC(=O)OC[C@H]1O[C@@H]([n+]2cccc(C#N)c2)[C@H](OC(C)=O)[C@@H]1OC(C)=O ; "con. HCl" ; NC(=O)c1ccc[n+]([C@@H]2O[C@H](CO)[C@@H](O)[C@H]2O)c1 ; [Cl-]]

To a solution of 4a (3.63 g 10 mmol) in HCl (40 mmol) in EtOH:H₂O (8:2) was stirred at 40° C. for 12 h. After completion, the solvent was removed under reduced pressure and the residue was diluted with acetone and the solid was filtered and dried to give crude NRCI, which was purified from acetone to give the pure amorphous solid of NRCI in 2.16 g, 85% yield as a white solid.

### Example 6

### Stage I: Chlorination of D-Ribose Tetraacetate (RTA) Using Anhydrous HCl

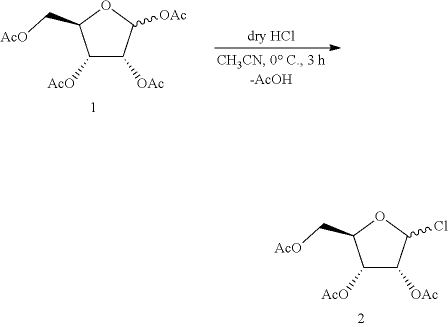 [SMILES: CC(=O)OC[C@H]1OC(OC(C)=O)[C@H](OC(C)=O)[C@@H]1OC(C)=O ; "dry HCl" ; CC(=O)OC[C@H]1OC(Cl)[C@H](OC(C)=O)[C@@H]1OC(C)=O]

To a stirred solution of D-Ribose tetraacetate (10 g, 31.5 mmol) in 25 mL acetonitrile (2.5 vol) in 250 mL round bottom flask at room temperature. The solution was cooled to 0° C. and then passed anhydrous HCl gas (1.83 g, 1.6 equiv) into the reaction mixture over 30 min. The resulting mixture was stirred at 0-5° C. for another 3 h. The progress of the reaction was monitored by TLC (1:1, EtOAc:hexane) and also by GC (IPC Limit: content of RTA by GC is NMT 5%). Stage II: Synthesis of tri-O-acetyl β-nicotinonitrile riboside chloride

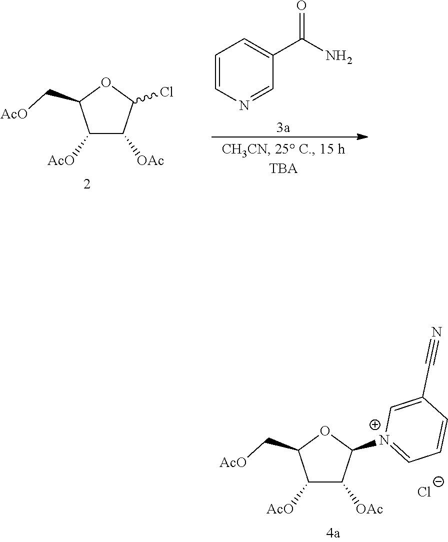

Upon complete conversion of stage 1, the nitrogen gas was purged into the reaction mixture for 10-15 min and charged 3-cyanopyridine (3.27 g, 1.0 equiv) in 10 mL acetonitrile (1 vol) at 0-5° C. and then added diisopropylethylamine (DIPEA, 4.0 g, 1.0 equiv) or tributylamine (TBA, 5.82 g 1.0 equiv) slowly at the same temperature. The resulting mixture was stirred at 25° C., over 15 hrs at the same temperature. The progress of the reaction was monitored by TLC (5:0.3:0.05 EtOAc/MeOH/triethylamine eluent); visualized the TLC plate with 10% H₂SO₄/MeOH. After complete disappearance of starting ribose tetraacetate and the appearance of a product as a single UV active spot, the solvent was removed and extracted the crude with 1:1 MeOH/hexane (5×100 mL). The n-hexane layer was separated and the methanol layer was concentrated to give the product 4a in 4.0 g, 35% yield as a colorless liquid, which was used in next step without any further characterization.

### Stage III: Hydrolysis/Deacetylation of 4a

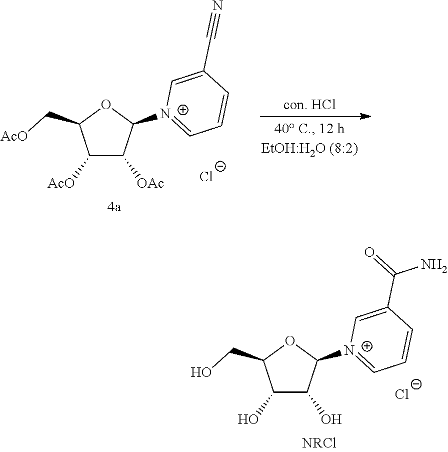 [SMILES: CC(=O)OC[C@H]1O[C@@H]([n+]2cccc(C#N)c2)[C@H](OC(C)=O)[C@@H]1OC(C)=O ; [Cl-] ; "con. HCl" ; NC(=O)c1ccc[n+]([C@@H]2O[C@H](CO)[C@@H](O)[C@H]2O)c1 ; [Cl-]]

To a solution of 4a (3.63 g. 10 mmol) in HCl (40 mmol) in EtOH:H₂O (8:2) was stirred at 40° C. for 12 h. After completion, the solvent was removed under reduced pressure and the residue was diluted with acetone and the solid was filtered and dried to give crude NRCI, which was purified from acetone to give the pure amorphous solid of NRCI in 1.7 g, 70% yield as a white solid.

### Industrial Advantages:

In the present invention, acetone has been used as a solvent for the first time for the chlorination of 1,2,3,5-tetra-O-acyl-D-ribofuranose and coupling reaction with nicotinamide. Moreover, acetone has been used for both the reaction as well as for purification in the synthesis of triacetyl-NRCI, in scheme 2.

Acetonitrile has been used as a solvent for the chlorination of 1,2,3,5-tetra-O-acyl-D-ribofuranose and subsequent reaction with 3-cyanopyridine, as shown in scheme 3.

Deacetylation of 2,3,5-triacetyl-NRCI has been performed for the first time using HCl in methanol or ethanol or isopropanol at temperature of 0-25° C., thereby avoided the requirement of cryogenic conditions. In this reaction, methanol or ethanol or isopropanol acts as a solvent for both the reaction and purification in the synthesis of NRCI.

The method is commercially viable due to the use of cost-effective solvents like acetone, methanol and ethanol and reagents like HCl for both chlorination of ribose tetraacetate (RTA) and deacetylation of 2,3,5-triacetyl-NRCI. The method is also feasible due to insitu reaction using solvent like acetone for both chlorination and coupling reaction with Nicotinamide, as shown in scheme 2 or acetonitrile as shown in scheme 3.

The use of acetone in the first step makes the method cost-effective and it is used as a solvent for the reaction and for the purification. In the second step, methanol or ethanol was used for the reaction and purification, which is contrary to the previous reports, wherein, multiple solvents were used for the reaction and purification.

The method is commercially feasible due to the insitu reaction using solvent like acetonitrile for both chlorination and coupling reaction with 3-cyanopyridine and reagents like HCl for both chlorination of ribosetetraacetate (RTA) and deacetylation of 2,3,5-triacetyl-NRCI, as shown in scheme 3.

The present processes are a two-step processes. In the entire process, only two solvents were used as reaction solvent and for purification.

Also, previous methods involve the use of anhydrous ammonia or dialkyl amines as deacetylating agents, in which acetamide or dialkyl amide used to be the by-product. Therefore, tedious work up and purification methods were required to remove acetamide or dialkyl amine and other by-products. However, in the present methods, anhydrous HCl was used as a deacetylating agent and hence the by-product formed during the deacetylation is ethyl acetate, which is volatile and environmentally friendly solvent and can be removed easily from the final product by simple work up.

In the previous methods, cryogenic conditions (−5 to −7° C.) were used for deacetylation; however, the deacetylation reaction in the present invention was performed at 0 to 25° C.

The present invention therefore provides cleaner reaction profiles; improved yields, high selectivity and purity and simplicity in the work-up and purification, thereby makes the processes cost effective and industrially scalable.

The first example of NRCI synthesis from ribose tetraacetate (D-RTA) and 3-cyanopyridine using TMSOTf or anhydrous HCl in acetonitrile or acetone is disclosed in the present invention, which is a novel approach for NRCI synthesis. There are several advantages of using 3-cyanopyridine in the synthesis of NRCI, which include operational simplicity, improved yields and high β-selectivity.

1. A process for the preparation of nicotinamide riboside chloride which process comprises
a) chlorinating ribose tetraacetate (RTA) by treating with dry HCl either in acetone or in acetonitrile followed by in situ coupling of chloro derivative either with nicotinamide or with 3-cyanopyridine respectively in the presence of an organic base to obtain tri-O-acetyl β-nicotinamide riboside chloride or tri-O-acetyl β-nicotinonitrile riboside chloride respectively; and
b) deacetylating the tri-O-acetyl β-nicotinamide riboside chloride or the tri-O-acetyl β-nicotinonitrile riboside chloride in the presence of HCl in an alcoholic solvent, to afford nicotinamide riboside chloride.

2. The process as claimed in claim 1, wherein, the process comprises
a) chlorinating ribose tetraacetate (RTA) by treating with dry HCl in acetone followed by in situ coupling of chloro derivative with nicotinamide in the presence of an organic base to obtain tri-O-acetyl β-nicotinamide riboside chloride; and
b) deacetylating the tri-O-acetyl β-nicotinamide riboside chloride in the presence of anhydrous HCl in an alcoholic solvent to afford nicotinamide riboside chloride.

3. The process as claimed in claim 1, wherein, the process comprises:
a) chlorinating ribose tetraacetate (RTA) by treating with dry HCl in acetonitrile followed by in situ coupling of chloro derivative with 3-cyanopyridine in the presence of an organic base to obtain tri-O-acetyl β-nicotinonitrile riboside chloride; and
b) deacetylating the tri-O-acetyl β-nicotinonitrile riboside chloride in the presence of HCl in an alcoholic solvent to afford nicotinamide riboside chloride.

4. The process as claimed in claim 1, wherein, the chlorination of 2,3,4,5-tetra-O-acetyl-D-ribose can also be performed in a solvent selected from the group consisting of methyl ethyl ketone, methyl isobutyl ketone and C4 to C6 ketonic solvents.

5. The process as claimed in claim 1, wherein, the chlorination reaction can be conveniently carried out at a temperature range of 0-5° C.

6. The process as claimed in claim 1, wherein, the reaction of step a) is carried out at a temperature range of 0° C. to 50° C.

7. The process as claimed in claim 1, wherein, the dry HCl or anhydrous HCl used in a solvent can be selected from the group consisting of freshly prepared dry HCl or anhydrous HCl absorbed in a solvent or commercially available dry or anhydrous HCl absorbed in a solvent, wherein, the solvents are selected from acetone, acetonitrile, methanol, ethanol and isopropanol.

8. The process as claimed in claim 1, wherein, the molar ratio of ribose tetraacetate to nicotinamide is in the ratio of 1:0.5 to 2.0.

9. The process as claimed in claim 1, wherein, the alcoholic solvent used in step b) is selected from the group consisting of methanol, ethanol, isopropyl alcohol, n-propyl alcohol and other C4 to C6 alcoholic solvents.

10. The process as claimed in claim 1, wherein, the organic base is selected from DIPEA (diisopropylethylamine), TBA or N-alkyl pyrrolidine.

11. The process as claimed in claim 1, wherein, the reaction of step b) can be performed at a temperature range of 0 to 25° C.

12. A process for the preparation of nicotinamide riboside chloride, which process comprises:
a) preparing tri-O-acetyl β-nicotinonitrile riboside triflate by treating ribose tetraacetate (RTA) with 3-cyanopyridine in the presence of TMSOTf in a solvent selected from dichloromethane or dichloroethane; and
b) hydrolyzing followed by deacetylation of tri-O-acetyl β-nicotinonitrile riboside triflate using HCl in ethanol/water to afford nicotinamide riboside chloride.

13. The process as claimed in claim 1, wherein, the nicotinamide riboside chloride thus obtained is in an amorphous form.
